(19) United States
(12) Patent Application Publication
(10) Pub. No.: US 2024/0191303 A1
(43) Pub. Date: Jun. 13, 2024

(54) METHODS FOR OPTIMIZING THE TREATMENT OF COLORECTAL CANCER

(71) Applicant: Brown University, Providence, RI (US)

(72) Inventors: Wafik S. El-Deiry, Barrington, RI (US); Lindsey Carlsen, Providence, RI (US)

(21) Appl. No.: 18/285,650

(22) PCT Filed: Apr. 5, 2022
(86) PCT No.: PCT/US2022/023506

§ 371 Date: Oct. 4, 2023

Related U.S. Application Data
(60) Provisional application No. 63/219,239, filed on Jul. 7, 2021.
(60) Provisional application No. 63/170,696, filed on Apr. 5, 2021.

(51) Int. Cl. C12Q 1/6886; A61K 31/282; A61K 31/4745; A61K 31/513; A61K 33/243

(52) U.S. Cl. CPC C12Q 1/6886; A61K 31/282; A61K 31/4745; A61K 31/513; A61K 33/243; C12Q 2600/106

(57) ABSTRACT

The invention provides a method for optimizing the treatment of colorectal cancer in a subject comprising the steps of extracting colorectal cancer cells from the subject, determining the transcriptional response in colorectal cancer cells across four or more clinically used chemotherapeutic drugs in terms of gene identity, magnitude of change, and p53 dependence, and determining the gene signature to determine the optimal course of treatment for the subject.

### TECHNICAL FIELD OF THE INVENTION

This invention generally relates to methods for optimizing the treatment of colorectal cancer.

### BACKGROUND OF THE INVENTION

Colorectal cancer (CRC) is the second leading cause of cancer deaths. Over 100,000 new cases may be diagnosed in the United States annually.

Chemotherapy with 5-fluorouracil (5-FU) combined with irinotecan (CPT-11) or oxaliplatin is a widely administered and generally effective treatment option for patients with colorectal cancer. Despite their efficacy and toxicity, these drugs' mechanisms remain incompletely understood, resulting in less optimal chemotherapy for patients.

There is a need in the biomedical art for methods for optimizing the treatment of colorectal cancer.

### SUMMARY OF THE INVENTION

The invention provides a method for optimizing the treatment of colorectal cancer in a subject. This method comprises the steps of extracting colorectal cancer cells from the subject, determining the transcriptional response in colorectal cancer cells across four or more clinically used chemotherapeutic drugs in terms of gene identity, the magnitude of change, and p53 dependence, and determining the gene signature to determine the optimal course of treatment for the subject.

While earlier work investigated cellular responses to chemotherapeutic agents individually, this method compares the transcriptomic and cytokine profiles of wild-type and p53^{-/-} colorectal cancer cells treated with these drugs and reports pan-drug, drug-specific, drug class-specific, p53-independent, and p53-dependent signatures. Downregulation of histone genes by 5-fluorouracil correlates with improved survival in colorectal cancer patients. Upregulation of FOS and ATF3 by oxaliplatin contributes to peripheral neuropathy. The use of the method identified BTG2 as a top gene upregulated by drugs.

In a first embodiment, the invention provides a database for optimizing the treatment of colorectal cancer. The gene signatures within this database unravel these mechanisms, aiding the selection of combination treatments and directing the development of more targeted therapies, which results in improved outcomes for this disease. The signatures may help clinicians understand the molecular basis of the response of specific colorectal tumors to given chemotherapeutic agents.

This dataset directly compares transcriptomic and cytokine responses of colorectal cancer cells to equitoxic doses of 5-fluorouracil, CPT-11, oxaliplatin, and cisplatin across p53 status. Thus, this dataset shows vast differences in the magnitude of the fold change of several genes and cytokines across the drug treatment groups.

In a second embodiment, the database is used by researchers to serve as a reference for p53 wild-type function when activated by clinically relevant drugs, enabling the development of p53-reactivating compounds. In other embodiments, the database is used by researchers to investigate drug-specific mechanisms and develop targeted therapies. This dataset permits evaluation of gene and cytokine responses similar across drugs, emphasizing or revealing their critical function in the p53-independent or p53-dependent cellular responses to chemotherapy in colorectal cancer, furthering the understanding of the mechanisms that mediate efficacy and toxicity in the treatment of colorectal cancer, providing guidance in combination treatment selection, insights for the development of targeted therapies, and prognostic markers for colorectal cancer patients based on the treatments they receive, and enabling investigation of specific transcripts or sets of transcripts within these signatures can uncover additional mechanisms of pan-drug, drug-specific, drug class-specific, p53-independent, and p53-dependent efficacy and toxicity.

In a third embodiment, the database is used in a clinical setting to develop targeted therapies for a subject afflicted with colorectal cancer. Tumor expression of gene signatures like that in the database after drug treatment may predict improved outcomes.

In one aspect, identifying synergistic and antagonistic mechanisms between drugs may suggest effective combination therapies. Drug class-specific mechanisms (such as platinum compounds) or unique drug activities can be identified from the database. Prevention of side effects may be possible by identifying mechanisms of drug-specific toxicity (both p53-dependent and p53-independent).

No existing database compares gene signatures in colorectal cancer across all chemotherapy drugs clinically used to treat colorectal cancer and across p53 status directly. Indirect comparison between databases introduces variables such as status of the cell line, treatment time, dose of the drug, and analysis method, making it difficult to make comparisons across drugs. Both unique drug effects, drug class effects, p53-dependent and p53-independent effects of clinically-used chemotherapeutics were used to treat colorectal cancer as far as efficacy and toxicity are helped by using the database and gene signatures.

Although others have looked at specific drug effects, this invention is the first to look across the most clinically used chemotherapeutics for colorectal cancer and identified novel and unique drug-specific gene signature effects, gene signature effects associated with drug classes such as platinum compounds, and p53-dependent versus p53-independent gene signatures relevant to chemotherapy drug efficacy and toxicity.

In a third embodiment, besides using the signatures to understand and predict the response or toxicity of the standard chemotherapeutic agents individually, the signatures may help explain the response or toxicity from standard combination chemotherapy regimens used to treat colorectal cancer.

In a fifth embodiment, the invention provides a method for optimizing the treatment of colorectal cancer in a subject, which comprises the steps of extracting colorectal cancer cells from the subject, determining the transcriptional response in colorectal cancer cells across four or more clinically used chemotherapeutic drugs in terms of gene identity, the magnitude of change, and p53 dependence, and determining the gene signature to determine the optimal course of treatment for the subject.

Other embodiments are also described and recited herein. Drug-specific mechanisms of efficacy or toxicity identified in these signatures can be targeted with combination therapies or the development of new targeted therapies. Together, the findings in this specification contribute to our understanding of the molecular bases of efficacy and toxicity of chemotherapeutic agents often used for the treatment of gastrointestinal (GI) cancers such as colorectal cancer.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 shows the filtering method used to create p53-dependent and p53-independent gene signatures for each drug. FC: fold change.

FIG. 2 shows transcripts highly induced by all drug treatments in a p53-dependent manner. (*) indicates a significant variation in the magnitude of change between drug treatments (standard deviation>0.7). 5-fluorouracil (5-FU) preferentially upregulated BAX in a p53-independent manner at this timepoint.

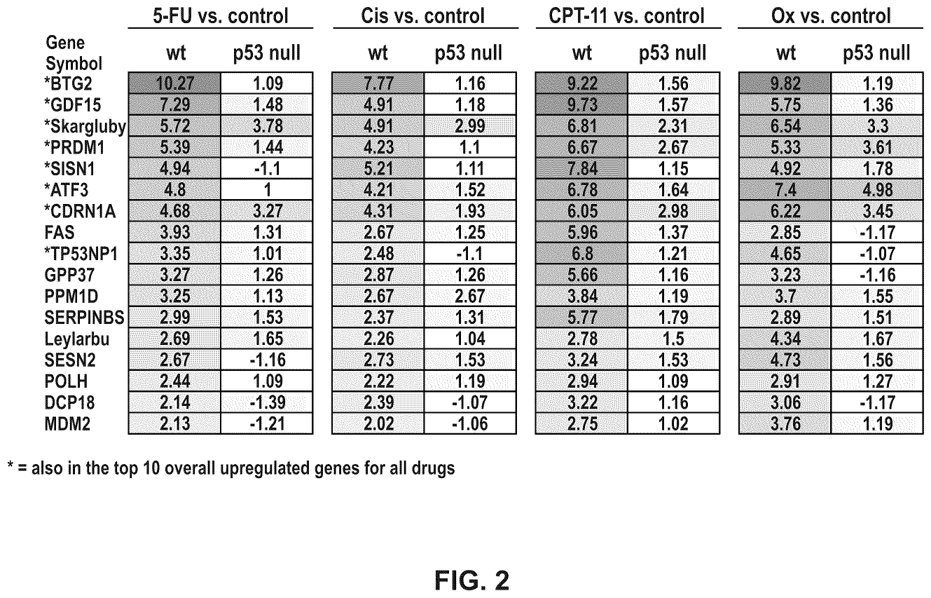

FIG. 3 shows that histone genes were uniquely down-regulated by 5-fluorouracil in a p53-independent manner. The experimental design used to create gene signatures was: HCT116 and HCT116 p53^{-/-} cells were treated with cisplatin, oxaliplatin, CPT-11, or 5-fluorouracil at their IC₅₀ for eight hours. RNA expression relative to an untreated control was measured using microarrays.

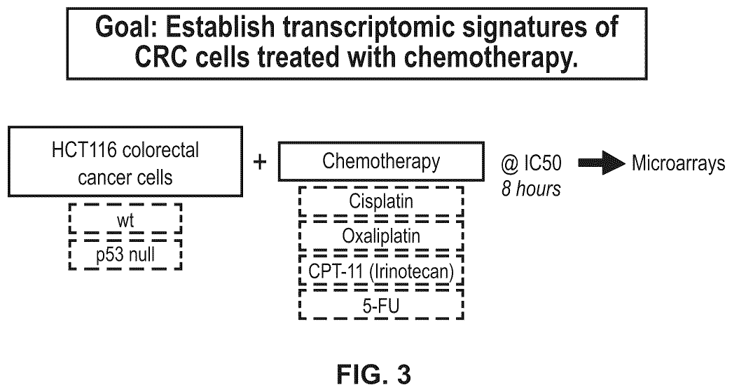

FIG. 4 is a TABLE showing the p53-dependent signatures and specific transcripts of interest regulated by all four drugs.

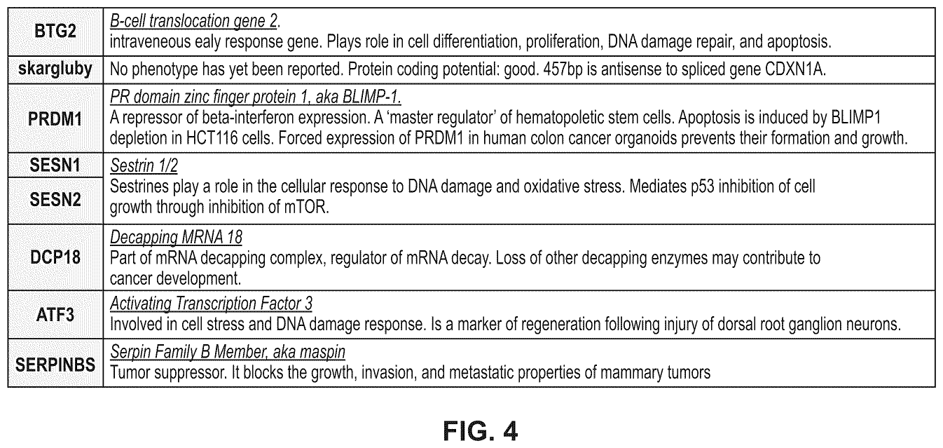

FIG. 5 is a TABLE showing the p53-dependent signatures, drug-specific transcripts of interest.

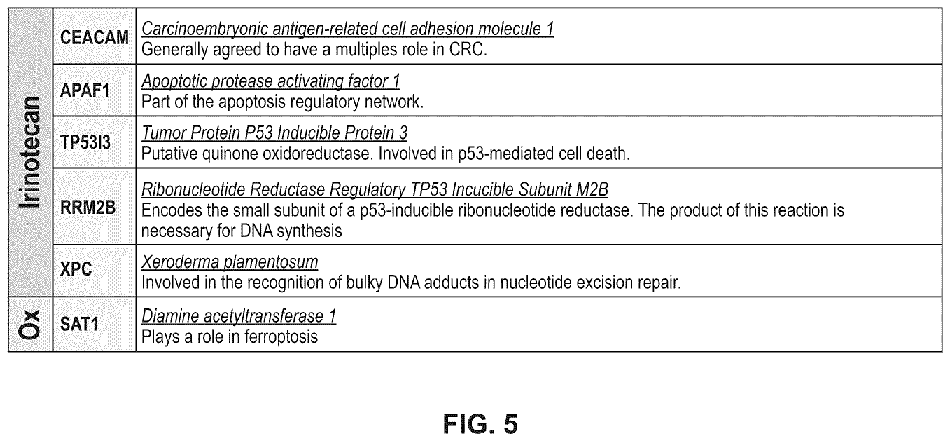

FIG. 6 is a bar graph showing p53-dependent signatures and drug-specific transcripts of interest.

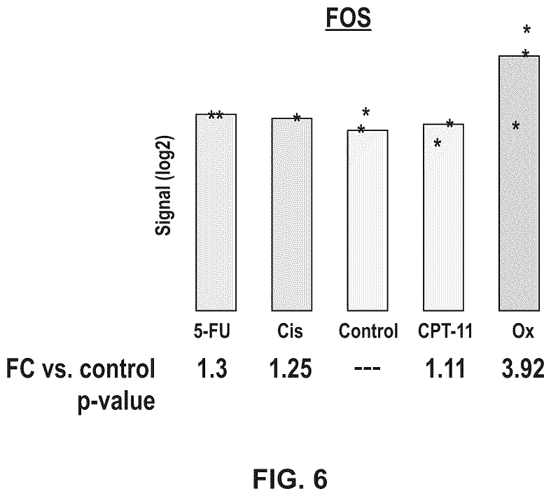

FIG. 7. Cytokine profiling reveals drug- and drug combination-specific induction of TRAILR2, IL-8, VEGF, and ferritin after 5-fluorouracil, CPT-11, oxaliplatin, and cisplatin treatment of human colorectal cancer cells. HCT116 and HCT116 p53^{-/-} cells were treated with the four drugs at their IC₅₀s, and combination treatment groups received two-three drugs, each at their IC₅₀ concentration, for forty-eight hours. Cytokine levels in the cell supernatants were measured with the Luminex 200 platform, and significant differences (p-value<0.05 by one-way ANOVA) between control and treated groups were calculated.

### DETAILED DESCRIPTION OF THE INVENTION

The subject innovation is now described concerning the drawings, wherein like reference numerals are used to refer to like elements throughout. In the following description, for an explanation, numerous specific details provide a thorough understanding of the present invention. However, it may be evident that the present invention may be practiced without these specific details. In other instances, well-known structures and devices are shown in block diagram form to describe the present invention. Aspects, modes, embodiments, variations, and features of the invention are described below in various levels of detail to provide a substantial understanding of the present invention.

### INDUSTRIAL APPLICABILITY

Identifying and exploiting specific mechanisms of efficacy or toxicity of individual chemotherapeutic drugs could help improve or predict gastrointestinal tumor outcomes. Prior publications described investigations of these drugs separately. RNA expression has been evaluated in patient samples after combination treatment. But these investigations involved several variables that make it difficult for persons having ordinary skill in the medical art to decipher mechanistic differences across drugs. Negrei et al., Front. Pharmacol., 7, 172 (2016); Del Rio et al., J. Clin. Oncol., 25, 773-80 (2007).

Direct comparison across drugs could elucidate novel drug-specific mechanisms, aiding the preclinical development of targeted therapies, refining existing compounds, and guiding informed combination therapies in the clinic. This analysis would also give clinicians a better understanding of the molecular basis of the response of colorectal cancer tumors exposed to these drugs.

Reactivation of wild-type (WT) p53 function holds therapeutic potential. However, little success has been made in this area. The p53 response is recognized in the medical art to vary across the tissue, cell type, drug type, and drug dose. The most important p53 targets for tumor suppression may differ across cancer types. This heterogeneity has yet not been used clinically.

An understanding of heterogeneity in the p53 response across chemotherapeutics used for colorectal cancer would enhance our understanding of what the most important p53 targets are in the treatment of colorectal cancer, personalize predictive/prognostic biomarkers for patients based on their p53 status, suggest potential combination therapies, and explain drug-specific efficacy or toxicity across p53 status. Due to considerable variation across studies seeking to identify p53 targets, evaluation of heterogeneity in the p53 response is likely best achieved by direct comparison across drugs. See Fischer, Oncogene, 36, 3943-56 (2017).

Heterogeneity in the p53 response to traditional chemotherapy is recognized, but its significance has yet to be used in the clinic. Further investigation could provide insight into the relative importance of individual p53 target genes and identify unique characteristics of specific drugs, guiding therapy selection.

Prognostic and predictive biomarkers are a cornerstone of precision oncology as their identification could help direct patients towards the most appropriate therapeutic interventions.

Based on prior publications, some transcripts can promote the anticancer effects of the drugs. Others may have a multiplex function or contribute primarily to toxicity. Future directions also include evaluating patient outcomes about basal and induced levels of transcripts within these signatures, which could provide clinical relevance and help guide the selection of transcripts for mechanistic in vitro and in vivo studies. Insights from this study may be exploited in the clinic in several ways. Establishing unique drug mechanisms may enable physicians to make informed decisions regarding combination therapies to promote efficacy or prevent toxicity.

And correlating specific genes or signatures with patient outcomes may enable prognostication for patients receiving certain drugs. These signatures and the biomarkers they represent may relate to other tumor types where these chemotherapy agents are used, demonstrating the potential of this study to have a broad impact across different types of cancer.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the biomedical art to which this invention belongs. A term's meaning provided in this specification shall prevail if any apparent discrepancy arises between the meaning of a definition provided in this specification and the term's use in the biomedical art.

The singular forms a, an, and the like include plural referents unless the context dictates otherwise. For example, a reference to a cell comprises a combination of two or more cells.

5-Fluorouracil (5-FU) has the definition provided by the National Cancer Institute. This antimetabolite inhibits thymidylate synthase (TS), which prevents the production of deoxythymidine monophosphate, which is essential for DNA replication and repair. Zhang, Yin, Xu, & Chen, 5 Molecules, 13, 1551-69 (2008). 5-Fluorouracil was combined with oxaliplatin and CPT-11 to improve outcomes in the clinic. Xie, Chen, & Fang, Signal Transduction Target Therapy. 5, 22 (2020). Questions remain regarding the exact mechanisms downstream of 5-fluorouracil-mediated thymidylate synthase inhibition.

Approximately or About means that a value or parameter is generally taken to include numbers that fall within a range of 5%, 10%, 15%, or 20% in either direction (greater than or less than) of the number unless otherwise stated or otherwise evident from the context (unless such number would be less than 0% or exceed 100% of a possible value). Reference to approximately or about a value or parameter includes (and describes) embodiments directed to that value or parameter. For example, a description referring to about X includes a description of X.

B-cell translocation gene 2 (BTG2) has the medical art-recognized meaning of a tumor suppressor that functions in the p53-dependent component of the DNA damage response, and its low expression correlates with more severe disease in breast and prostate cancer. Yuniati, Scheijen, van der Meer, & van Leeuwen. J. Cell. Physiol., 234, 5379-89 (2019). BTG2 likely functions in contributing efficacy of these drugs for the treatment of colorectal cancer. BTG2 was identified as a top gene upregulated by all four drugs (5-FU, CPT-11, oxaliplatin, and cisplatin). This upregulation of BTG2 by compounds with different mechanisms and p53 induction profiles emphasizes BTG2 importance in the cellular response to DNA damage. It reveals its critical function in mediating the p53 response to chemotherapy used to treat colorectal cancer. As BTG2 upregulation is p53-dependent, particular focus should be dedicated to this transcript when designing p53-reactivating therapies and further analyzing the relationship to drug response and outcomes in patient cohorts with colorectal cancer.

Beneficial or desired clinical results include, but are not limited to, alleviation of one or more symptoms, diminishment of the extent of the deficit, stabilized (i.e., not worsening) state of a tumor or malignancy, delay or slowing of tumor growth or metastasis, and an increased lifespan as compared to that expected absent treatment.

Benign or non-malignant has the medical art-recognized meaning of tumors that may grow larger but do not spread to other parts of the body. Benign tumors are self-limited and rarely invade or metastasize.

Cancer Cell or Tumor Cell has the medical art-recognized meaning of an individual cell of a cancerous growth or tissue. A cancer cell is a cancerous, pre-cancerous, or transformed cell, either in vivo, ex vivo, or in tissue culture, with spontaneous or induced phenotypic changes that do not necessarily involve the uptake of new genetic material. Although transformation can arise from infection with a transforming virus and incorporation of new genomic nucleic acid or uptake of exogenous nucleic acid, it can also occur spontaneously or following exposure to a carcinogen, mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor-specific markers, invasiveness or metastasis, and tumor growth in suitable animal hosts such as nude mice.

Cancer has the medical art-recognized meaning of a class of diseases or conditions in which abnormal cells divide without control and can invade nearby tissues. Cancer cells can also spread to other body parts through the blood and lymph systems. In some embodiments, the cancer is primary cancer. In some embodiments, the cancer is malignant.

Carcinoma has the medical art-recognized meaning of cancer that begins in the skin or in tissues that line or cover internal organs.

Central nervous system cancers have the medical art-recognized meaning of cancers that begin in the brain and spinal cord tissues.

Colorectal Cancer (CRC) has the medical art-recognized meaning of a disease in which cells in the colon or rectum grow out of control. Sometimes it is called colon cancer, for short. See Division of Cancer Prevention and Control, United States Centers for Disease Control and Prevention.

Complete inhibition is a 100% inhibition as compared to a reference level.

Comprising means that other elements can also be present in addition to the defined elements presented. Using comprising indicates inclusion rather than limitation.

Consisting essentially of means those elements required for a given embodiment. The term permits additional elements that do not materially affect the basic and functional characteristics of that embodiment of the invention.

Consisting of means compositions, methods, and respective components thereof, exclusive of any element not recited in that description of the embodiment.

CPT-11 (camptothecin-11, generic name: Irinotecan, trade name: Camptosar®) is a topoisomerase inhibitor that causes cytotoxic protein-linked DNA breaks. CPT-11 and oxaliplatin have strikingly different efficacy and toxicity profiles despite identical primary targets. These drugs have medical art-recognized anticancer activities that extend beyond those driven by their primary target. Alcindor & Beauger, Curr. Oncol., 18, 18-25 (2011); Bailly, Pharmacol. Res., 148, 104398 (2019); and Wyatt & Wilson, 3rd, Cell. Mol. Life Sci., 66, 788-99 (2009).

Decrease, Reduced, Reduction, or Inhibit has the medical art-recognized meaning of a decrease by a statistically significant amount. In some embodiments, reduce, reduction or decrease or inhibit typically means a decrease by at least 10% as compared to a reference level (e.g., the absence of a treatment or agent) and can include more significant decreases, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. Reduction or inhibition does not encompass a complete inhibition or reduction compared to a reference level. A decrease can be preferably down to a level accepted as within the range of normal for an individual without a disorder.

Effective Amount and Therapeutically-Effective Amount have the medical art-recognized meaning of an amount sufficient to prevent or ameliorate a manifestation of the disease or medical condition, such as colorectal cancer. Many ways are known in the biomedical art to determine the effective amount for an application. For example, pharmacological methods for dosage determination can be used in the therapeutic context. In therapeutic or prophylactic applications, the amount of a composition administered to the subject depends on the type and severity of the disease and the characteristics of the individual, such as general health, age, sex, body weight, tolerance to drugs, and on the degree, severity, and type of disease. Persons having ordinary skill in the biomedical art can determine appropriate dosages depending on these and other factors. The compositions can also be administered combined with one or more additional therapeutic compounds.

Engineered means the aspect of having been manipulated by the hand of man. For example, a polypeptide is engineered when at least one aspect of the polypeptide, e.g., its sequence, has been manipulated by the hand of man to differ from the aspect as it exists in nature. For example, an antibody, antibody reagent, antigen-binding portion thereof, CAR or bispecific antibody is engineered when the sequence of the antibody, antibody reagent, antigen-binding portion thereof, CAR or bispecific antibody is manipulated by the hand of man to differ from the sequence of an antibody as it exists in nature. As is common practice and is understood by those in the biomedical art, the progeny of an engineered cell is typically still called engineered even though the actual manipulation was performed on a prior entity.

Expression Products include RNA transcribed from a gene and polypeptides obtained by translation of mRNA transcribed from a gene.

Expression Vector means a vector that directs the expression of an RNA or polypeptide from sequences linked to transcriptional regulatory sequences on the vector. The sequences expressed are often but are not necessarily heterologous to the cell. An expression vector may comprise additional elements. For example, the expression vector may have two replication systems, thus maintaining it in two organisms, such as human cells for expression and a prokaryotic host for cloning and amplification. The term expression means the cellular processes involved in producing RNA and proteins and, as appropriate, secreting proteins, including, where applicable, but not limited to, for example, transcription, transcript processing, translation, protein folding, modification, and processing.

Functional Fragment has the medical art-recognized meaning of a fragment or segment of a peptide that retains at least 50% of the wild-type reference polypeptide's activity according to the assays described herein. A functional fragment can comprise conservative substitutions of the sequences disclosed herein.

Gastrointestinal (GI) Cancer or Gastric Cancer has the medical art-recognized meaning of malignant conditions of the gastrointestinal tract (GI tract) and accessory organs of digestion, including the esophagus, stomach, biliary system, pancreas, small intestine, large intestine, rectum, and anus. See United States National Cancer Institute. Colorectal cancer is a subset of gastrointestinal cancers.

Gene has the medical art-recognized meaning of the nucleic acid sequence transcribed (DNA) to RNA in vitro or in vivo when operably linked to appropriate regulatory sequences. The gene might include regions preceding and following the coding region, e.g., 5′ untranslated (S′UTR) or leader sequences and 3′ UTR or trailer sequences, and intervening sequences (introns) between individual coding segments (exons).

Growth differentiation factor 15 (GDF15) has the medical art-recognized meaning. This TGF-beta ligand likely promotes epithelial to mesenchymal transition and metastasis in colorectal cancer through activation of Smad2 and Smad3 pathways. Evaluation of GDF15 in patients in several studies revealed that high levels correlate with increased chances of metastasis, lower overall survival, and weight loss. GDF15 neutralization in non-human primates decreased cisplatin-induced weight. Conversely, 5-fluorouracil-resistant colorectal cancer cells express lower levels of GDF15 compared to 5-fluorouracil-sensitive cells and transient expression of GDF15 restores sensitivity, suggesting this gene functions in 5-fluorouracil-mediated cell death. GDF15 likely has a multiplex function in response to chemotherapy

Increased, Increase, Enhance, or Activate has the medical art-recognized meaning of an increase by a statically significant amount. In some embodiments, the terms increased, increase, enhance, or activate can mean an increase of at least 10% as compared to a reference level, for example, an increase of at least about 20%, or at least about 30%, or at least about 40%, or at least about 50%, or at least about 60%, or at least about 70%, or at least about 80%, or at least about 90% or including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or more significant as compared to a reference level. In the context of a marker or symptom, an increase is a statistically significant increase in such a level.

Isolated or Partially Purified has the medical art-recognized meaning of a nucleic acid or polypeptide separated from at least one other component (e.g., nucleic acid or polypeptide) that is present with the nucleic acid or polypeptide as found in its natural source or that would be present with the nucleic acid or polypeptide when expressed by a cell or secreted with secreted polypeptides. A chemically synthesized nucleic acid or polypeptide or one synthesized using in vitro transcription/translation is isolated.

Leukemia has the medical art-recognized meaning of cancer that starts in blood-forming tissue such as the bone marrow and causes many abnormal blood cells to be produced and enter the blood.

Long-Term Administration has the medical art-recognized meaning that the therapeutic agent or drug is administered for at least twelve weeks. This administration includes that the therapeutic agent or drug is administered such that it is effective over, or for, a period of at least twelve weeks and does not necessarily imply that the administration itself takes place for twelve weeks, e.g., if sustained release compositions or long-acting therapeutic agent or drug is used. Thus, the subject is treated for at least 12 weeks. Often, long-term administration is for at least 4, 5, 6, 7, 8, 9 months or more, or for at least 1, 2, 3, 5, 7, or 10 years or more. The administration of the compositions contemplated herein may be carried out in any convenient manner, including by aerosol inhalation, injection, ingestion, transfusion,

Lymphoma and Multiple Myeloma are cancers that begin in the cells of the immune system.

Malignant has the medical art-recognized meaning of a cancer in which a group of tumor cells displays one or more of uncontrolled growth (i.e., division beyond normal limits), invasion (i.e., intrusion on and destruction of adjacent tissues), and metastasis (i.e., spread to other locations in the body via lymph or blood).

Metastasize has the medical art-recognized meaning that the spread of cancer from one part of the body to another. A tumor formed by cells that have spread is called a metastatic tumor or a metastasis. The metastatic tumor contains cells like those in the original (primary) tumor.

Nucleic Acid or Nucleic Acid Sequence has the medical art-recognized meaning of any molecule, preferably a polymeric molecule, incorporating units of ribonucleic acid, deoxyribonucleic acid, or an analog thereof. The nucleic acid can be single-stranded or double-stranded. A single-stranded nucleic acid can be one nucleic acid strand of a denatured double-stranded DNA. Alternatively, it can be a single-stranded nucleic acid not derived from double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

Or means and/or. The term and/or as used in a phrase such as A and/or B herein includes both A and B; A or B; A (alone); and B (alone). Likewise, the term and/or as used in a phrase such as A, B, and/or C encompasses each embodiment: A, B, and C; A, B, or C; A or C; A or B; B or C; A and C; A and B; Band C; A (alone); B (alone); and C (alone).

Oxaliplatin and its analogue cisplatin, which is not used to treat colorectal cancer, are platinum-based therapeutics that damage DNA via inter-strand and intra-strand crosslinks. Despite identical primary targets, cisplatin and oxaliplatin have strikingly different efficacy and toxicity profiles, including oxaliplatin-specific peripheral neuropathy that occurs in 30-50% of patients. These drugs have medical art-recognized anticancer activities that extend beyond those driven by their primary target. Alcindor & Beauger, Curr. Oncol., 18, 18-25 (2011); Bailly, Pharmacol. Res., 148, 104398 (2019); and Wyatt & Wilson, 3rd, Cell. Mol. Life Sci., 66, 788-99 (2009).

p53 has the medical art-recognized meaning. See Fischer, Oncogene, 36, 3943-56 (2017). TP53 is the most frequently mutated gene in cancer and is mutated in ˜50% of colorectal cancer patients. The encoded protein, p53, is a transcription factor activated by cell stressors such as DNA damage, oncogenic signaling, and hypoxia. p53 responds by activating its target genes which mediate cell fates relevant to the response to chemotherapy, including apoptosis, cell cycle arrest, and DNA repair, among others. p53 mutations generally occur late in colorectal cancer disease progression and result in an increased lymphatic and vascular invasion, chemo-resistance, and a decline in prognosis. While much research has been done on p53 and its role in the DNA damage response and sensitivity to various chemotherapeutic agents, the clinical applicability has mainly remained underdeveloped. There is old evidence that radiotherapy for wild-type p53-expressing rectal cancer is associated with better patient outcomes than when p53 is mutated. There is also preclinical evidence that has associated p53 status with cell death and chemo-sensitivity after treatment of colorectal cancer cells with 5-fluorouracil. There is a medical art-recognized tissue specificity of the p53 response in vivo. Differences in how DNA damaging agents engage the p53 pathway leading to various outcomes such as different p53 phosphorylations or cellular phenotypes such as growth arrest, DNA repair, or cell death.

Protein and Polypeptide are used interchangeably herein to designate a series of amino acid residues connected by peptide bonds between the alpha-amino and carboxy groups of adjacent residues. The terms protein and polypeptide refer to a polymer of amino acids, including modified amino acids, e.g., phosphorylated, glycated, glycosylated, etc., and amino acid analogs, regardless of their size or function. Protein and polypeptide often mean relatively large polypeptides, whereas the term peptide often means small polypeptides, but the usage of these terms in biomedical art overlaps. The terms protein and polypeptide are used interchangeably herein when referring to a gene product and fragments thereof. Thus, exemplary polypeptides or proteins include gene products, naturally occurring proteins, homologs, orthologs, paralogs, fragments, and other equivalents, variants, fragments, and analogs of the foregoing. Variants (naturally occurring or otherwise), alleles, homologs, conservatively modified variants, or conservative substitution variants of any particular polypeptides described are encompassed. As to amino acid sequences, a person having ordinary skill in the biomedical art recognizes that individual substitutions, deletions, or additions to a nucleic acid, peptide, polypeptide, or protein sequence which alters a single amino acid or a small percentage of amino acids in the encoded sequence are a conservatively modified variant where the alteration results in the substitution of amino acid with chemically similar amino acid and retains the desired activity of the polypeptide. Such conservatively modified variants also do not exclude polymorphic variants, interspecies homologs, and alleles consistent with the disclosure. In some embodiments, the variant is conservatively modified. Conservative substitution variants can be obtained by mutations of native nucleotide sequences, for example.

Purified or Substantially Purified means an isolated nucleic acid or polypeptide at least 95% by weight of the subject nucleic acid or polypeptide, including at least 96%, at least 97%, at least 98%, at least 99% or more. In some embodiments, the antibody, antigen-binding portion thereof, or chimeric antigen receptor (CAR) described herein is isolated. The antibody, antibody reagent, antigen-binding portion thereof, or CAR described herein is purified in some embodiments.

Sarcoma has the medical art-recognized meaning of cancer that begins in bone, cartilage, fat, muscle, blood vessels, or other connective or supportive tissue.

Skargluby has the medical art-recognized meaning. This transcript is “identified by AceView,” meaning that they have an unknown function and, sometimes, have unknown coding potential. After splicing, skargluby is antisense to 457 base pairs of CDKN1A (coding for p21WAF1).

Statistically Significant or Significantly means statistical significance and generally means a two standard deviation (2SD) or more significant difference.

Subject In Need Of Treatment for a particular condition can be a subject having that condition, diagnosed as having that condition, or at risk of developing that condition.

Subject means a mammal, including but not limited to a dog, cat, horse, cow, pig, sheep, goat, chicken, rodent, or primate. Subjects can be house pets (e.g., dogs, cats), agricultural stock animals (e.g., cows, horses, pigs, chickens, etc.), laboratory animals (e.g., mice, rats, rabbits, etc.), but are not so limited. Subjects include human subjects. The human subject may be a pediatric, adult, or geriatric subject. The human subject may be of either sex.

Subject That Has a Cancer or Subject That Has a Tumor has the medical art-recognized meaning of a subject having objectively measurable cancer cells present in the subject's body. This definition includes malignant, actively proliferative cancers and potentially dormant tumors or micrometastases. Cancers that migrate from their original location and seed other vital organs can eventually lead to the subject's death through the functional deterioration of the affected organs. Hemopoietic cancers, such as leukemia, can out-compete the regular hemopoietic compartments in a subject, leading to a hemopoietic failure (in anemia, thrombocytopenia, and neutropenia), ultimately causing death. A subject can have been diagnosed with or identified as suffering from or having a condition needing treatment (e.g., cancer) or one or more complications related to such a condition, and optionally, but need not have already undergone treatment for a condition or the one or more complications related to the condition. A subject can also not have been diagnosed as having a condition needing treatment or one or more complications related to such a condition. For example, a subject can exhibit one or more risk factors for a condition or one or more complications related to a condition or a subject who does not show risk factors.

Treat, Treatment, Treating, or Amelioration of a Disease, Disorder, or Medical Condition has the medical art-recognized meaning of therapeutic treatments for a condition. The object is to reverse, alleviate, ameliorate, inhibit, slow down or stop the progression or severity of a symptom or condition. The term treating includes reducing or alleviating at least one adverse effect or symptom of a condition. Treatment is generally effective if one or more symptoms or clinical markers are reduced. Treatment is effective if the progression of a condition is reduced or halted. Treatment includes not just the improvement of symptoms or markers but also a cessation or at least slowing of progress or worsening of symptoms expected absent treatment.

Tumor has the medical art-recognized meaning of swelling or lesion formed by an abnormal growth of cells, which may be benign, pre-malignant, or malignant. Most cancer cells form tumors, but some, e.g., leukemia, do not necessarily form tumors. The terms cancer (cell) and tumor (cell) are used interchangeably for those cancer cells that form tumors.

Variant means a polypeptide substantially homologous to a native or reference polypeptide but with an amino acid sequence different from that of the native or reference polypeptide because of one or a plurality of deletions, insertions, or substitutions. Variant polypeptide-encoding DNA sequences encompass sequences that comprise one or more additions, deletions, or substitutions of nucleotides compared to a native or reference DNA sequence but encode a variant protein or fragment thereof retains activity. Many PCR-based site-specific mutagenesis approaches are known in the biomedical art and can be applied by the ordinarily skilled artisan.

Vector. A vector can comprise a nucleic acid encoding a polypeptide (e.g., an antibody or antibody reagent). A nucleic acid sequence encoding a polypeptide or any module thereof can be operably linked to a vector. A vector can include but is not limited to a cloning vector, an expression vector, a plasmid, phage, transposon, cosmid, chromosome, virus, virion, etc.

Unless otherwise defined herein, scientific and technical terms used with the present application shall have the meanings commonly understood by those of ordinary skill in the biomedical art to which this disclosure belongs. This invention is not limited to the particular methodology, protocols, reagents, etc., described herein and can vary. The terminology used herein is to describe specific embodiments only and is not intended to limit the scope of the present invention, which is defined solely by the claims. Definitions of standard terms in immunology and molecular biology can be found in The Merck Manual of Diagnosis and Therapy (1); The Encyclopedia of Molecular Cell Biology and Molecular Medicine (2); Molecular Biology and Biotechnology: a Comprehensive Desk Reference (3); Immunology (4); Janeway's Immunobiology (5); Lewin's Genes XI (6); Molecular Cloning: A Laboratory Manual (7); Basic Methods in Molecular Biology (8); Laboratory Methods in Enzymology (9); Current Protocols in Molecular Biology (CPMB) (10); Current Protocols in Protein Science (CPPS) (11); and Current Protocols in Immunology (CPI) (12).

Unless otherwise defined herein, scientific and technical terms used with this application shall have the meanings commonly understood by persons having ordinary skill in the biomedical art. This invention is not limited to the particular methodology, protocols, reagents, etc., described herein and as such can vary.

The disclosure described herein does not concern a process for cloning humans, processes for modifying the germ line genetic identity of humans, uses of human embryos for industrial or commercial purposes, or processes for modifying the genetic identity of animals likely to cause them suffering with no substantial medical benefit to man or animal, and animals resulting from such processes.

Guidance from Materials and Methods

Persons having ordinary skill in the biomedical art can use these materials and methods as guidance to predictable results when making and using the invention.

Cell lines and culture conditions. HCT116 and HCT116 p53^{-/-} human colorectal cancer cells were grown in McCoy's 5A medium supplemented with 10% fetal bovine serum (FBS) and 1% penicillin/streptomycin at 37 degrees, 5% CO₂. Cells were tested to ensure that they were mycoplasma free.

Establishing IC₅₀ doses. HCT116 and HCT116 p53^{-/-} cells were treated with doses ranging from 0.08-80 μM of 5-fluorouracil, CPT-11, oxaliplatin, or cisplatin for seventy-two hours in a 96-well plate. Cell viability was measured using the CellTiterGlo assay (Promega G7570), and the IC₅₀ dose was established from the resulting dose-response curve.

Western blots. Western blot was used to confirm a treatment-induced increase in p53 and investigate p53 target heterogeneity at the protein level. A total of 5×10⁵ HCT116 and HCT116 p53^{-/-} cells were plated in a 6-well plate and incubated for twelve-sixteen hours before being treated with 5-fluorouracil, CPT-11, oxaliplatin, or cisplatin at their respective IC₅₀ doses for several time points ranging from 1-48 hours. Proteins were extracted from cells with RIPA buffer containing protease inhibitor. Denaturing sample buffer was added, samples were boiled at 95° C. for ten minutes, and an equal amount of protein lysate was electrophoresed through 4-12% SDS-PAGE gels (Invitrogen) then transferred to PVDF membranes. The PVDF membrane was blocked with 5% non-fat milk (Sigma) in 1×TTBS. The primary antibodies were incubated with the transferred PVDF membrane in a blocking buffer at 4° C. overnight. Antibody binding was detected on PVDF with appropriate Pierce HRP-conjugated secondary antibodies by the Syngene imaging system. Invitrogen goat anti-rabbit IgG (H+L) secondary antibody, HRP #31460, and goat anti-mouse IgG (H+L) secondary antibody, HRP #31430, were diluted 1:5000 in 2.5% non-fat milk.

Microarrays. Total cellular RNA was isolated, and Clariom™ D microarrays were used to measure changes in RNA expression relative to untreated cells. A total of 7×10⁵ HCT116 and HCT116 p53^{-/-} cells were plated in a 6-well plate and incubated for twelve-sixteen hours before being treated with 5-fluorouracil, CPT-11, oxaliplatin, or cisplatin at their respective IC₅₀ doses for eight hours. Cell pellets were collected and split into two tubes, one for western blot analysis and one for RNA extraction. Samples were randomized, and RNA was isolated (Qiagen 74134) in five batches to ensure a high-quality RNA product. Acceptable RNA concentration and quality were verified with Nanodrop and Bioanalyzer measurements. Clariom D Microarrays (ThermoFisher 902923) were conducted according to the manufacturer's protocol in two batches on randomized samples to limit batch effects.

TCGA analysis. The publicly available computational tool cBioPortal was used to analyze TCGA data. Unless otherwise indicated, the Colorectal Adenocarcinoma PanCancer Atlas database (containing 592 total samples with RNA-seq data) was used. Differences in overall survival were evaluated between groups with high or low (>1 and <−1 standard deviation from the mean, respectively) basal expression of specific transcripts in the tumors despite TP53 status and in patients with TP53 wild-type tumors only. Log-transformed mRNA expression z-scores were compared to the expression distribution of all samples. z-score threshold=±2. A logrank test p-value under 0.05 (was considered significant, while a p-value between 0.05-0.2 (red border) was considered of potential interest. Both were included in the prognostic signatures.

Cytokine profiling. A total of 4×10⁴ HCT116 or HCT116 p53^{-/-} cells were plated per well of a 24-well plate and incubated twelve-sixteen hours before treatment with 55-fluorouracil, CPT-11, oxaliplatin, cisplatin, or various combination treatments at the appropriate IC₅₀ (combination treatments received each drug at their IC₅₀). Cell supernatants were collected at forty-eight hours after treatment and stored at −20° C. Samples were prepared and run in triplicate on a Luminex 200 Instrument (R&D LX200-XPON-RUO). Sample preparation was conducted, and instrument settings were selected based on the Human Magnetix Luminex Assay (R&D LXSAHM) protocol.

Statistical analysis. TAC software was used to calculate fold changes and statistical significance. Microarrays Applied Biosystems Transcriptomic Analysis Console (TAC) software was used to calculate fold-changes in gene expression relative to the untreated control cells. p-values<0.05 were considered significant.

Cytokine profiling. Results were analyzed in GraphPad prism, and statistical significance across drugs was determined separately for wild-type and ^{-/-} cells with a one-way ANOVA. p-values<0.05 were considered significant.

The following EXAMPLES are provided to illustrate the invention and shall not limit the scope of the invention.

### Example 1

### The Transcriptional Response in Colorectal Cancer Cells Varies Across Four Clinically Used Chemotherapeutic Drugs in Terms of Gene Identity, the Magnitude of Change, and p53 Dependence

Wild-type or p53 null HCT116 colorectal cancer cells were treated with oxaliplatin, cisplatin, CPT-11 (irinotecan), or 5-fluorouracil (5-FU) at an IC₅₀ dose for eight hours in triplicate. p53-dependent genes upregulated (fold change>2) by each drug were identified, revealing BTG2 as the top upregulated gene after oxaliplatin, cisplatin, and 5-fluorouracil treatment. Nine genes appeared highly induced in all four gene lists (BTG2, FAS, TP53INP1, GPR87, POLH, DCP1B, MDM2, PDGFA, and PHLDA3). Variations in the magnitude of change across drugs were noted for several, including BTG2, FAS, TP53INP1, GPR87, and MDM2.

Heterogeneity in the magnitude of change of p53 targets was observed by Western blot. These Western blots showed differential upregulation of p21 and DRS across drug treatment conditions. The lists contained several genes unique to each treatment, such as BAX, which showed slight preferential upregulation by 5-fluorouracil. Evaluation of the p53-independent transcriptional regulation revealed a strikingly significant and unique regulation of histone modifications by 5-fluorouracil compared to the other treatments.

Beyond these specific genes, variation in the magnitude of change and identifying the unique targets after each drug treatment highlight potentially important mechanistic differences between the chemotherapeutic drugs. The appearance of nine highly induced transcripts across all four drug treatments indicates their importance and the contribution of the p53 response to the molecular effects of chemotherapy.

Heterogeneity in the p53-dependent and p53-independent responses to 5-fluorouracil, cisplatin, CPT-11 (irinotecan), and oxaliplatin highlights important mechanistic differences between them.

### Example 2

Colorectal Cancer Gene Signatures. p53-Dependent or p53-Independent, and Clinical Outcomes with Specific Drugs Used Clinically to Treat Colorectal Cancer

EXAMPLE objectives. In colorectal cancer, several therapeutic agents are used, including 5-fluorouracil, irinotecan, and oxaliplatin, and several biologic agents. Specific genes have been associated with toxicity from 5-fluorouracil (such as specific DPD variants) or irinotecan (such as UGT1A1). Other alterations such as RAS gene mutations (KRAS or NRAS) have been associated with resistance to anti-EGFR therapy. Additional insights into the molecular basis of the response of colorectal cancer to various therapeutics would be helpful for understanding not only the p53-dependent mechanisms but also the p53-independent effects of drugs associated with sensitivity of colorectal cancer cells with mutations in p53.

The inventors' preclinical efforts involved profiling gene expression changes before and after exposure of human colorectal cancer cells to agents used to treat colorectal cancer (CRC) in isogenic cells with wild-type p53 or null for p53. The evidence from gene profiling of colorectal cancer cell lines treated with different anti-colorectal cancer drugs suggests genes and gene signatures of interest and relevance to drug sensitivity.

This EXAMPLE tests how tumor expression of specific transcripts or groups of transcripts related to specific drug exposure such as 5-fluorouracil, irinotecan, or oxaliplatin impacts on clinical outcomes for patients who received the drugs and the p53 status of the tumors.

The top gene identified was BTG2. Regarding dependence on p53 and degree of upregulation, there was a clear association between high expression and favorable patient survival but only for the tumors with wild-type p53. This supports the working plan. Basal expression of p53-activated genes identified from the cell line profiling could be predictive of long-term patient outcomes for patients with tumors with wild-type p53. In such tumors, exposure to the chemotherapeutic agents could leads to upregulation of genes such as BTG2 and others have been identified that may be involved in the therapeutic response and that may associate with patient outcomes.

The working plan can increase knowledge about colorectal cancer cell sensitivity to specific agents used to treat colorectal cancer and may allow prediction of a greater likelihood of response in colorectal cancer tumors based on their p53 status. The analysis can shed light on the unique mechanisms of drugs used to treat colorectal cancer, including about their efficacy or toxicity. Specific genes or signatures may allow prognostication for patients receiving certain drugs and for to other tumor types where the chemotherapy agents are used.
Task #1. Identify cohorts of patients with colorectal cancer tumors with wild-type p53 or mutant p53 that were treated with 5-fluorouracil, irinotecan, or oxaliplatin, with RNA-seq data.
Task #2. Determine the associations between low and high expression of specific (p53-dependent) transcripts or groups of transcripts as a signature and overall survival in patients with advanced colorectal cancer, regardless of treatment and as a function of specific drugs used.
Task #3. Evaluate the impact of wild-type p53 versus mutant p53 status in colorectal cancers about the observed clinical outcomes (patient survival) as determined by low and high expression of specific p53-dependent transcripts or signatures (groups of transcripts).
Task #4. Determine the relationship between identified p53-independent transcripts induced by drugs used to treat colorectal cancer (those induced across drugs or unique to certain drugs).
Task #5. Determine whether the identified relationships are altered by right versus left-sided tumors or as a function of age. The evidence from gene profiling of colorectal cancer cell lines treated with different anti-colorectal cancer drugs suggests genes and gene signatures of interest and relevance to drug sensitivity.
Task #6. Evaluate how tumor expression of specific transcripts or groups of transcripts related to specific drug exposure impacts on clinical outcomes for patients who received the drugs. Some transcripts should impact outcomes in tumors with wild-type (WT)-p53 versus p53-independent; 5-fluorouracil only; oxaliplatin+5-fluorouracil versus irinotecan+5-fluorouracil versus 5-fluorouracil; single genes or gene signatures (RNA-seq data) versus outcomes attributed to specific drugs or in treated patients (combinations).
Task #7. Evaluate the impact on outcomes of signatures relevant to all the drugs versus ones unique to specific drugs about outcomes.
Task #8. Evaluate the p53-dependence of the gene signature vs. outcomes in tumors with wild-type p53 vs. loss or mutation of p53.

### Example 3

### Drug-Specific Variability in the Kinetics of the p53 Response to 5-Fluorouracil, CPT-11, Oxaliplatin, and Cisplatin in Colorectal Cancer Cells

To show heterogeneity in the p53 response to drugs used to treat colorectal cancer, HCT116 and HCT116 p53^{-/-} cells were treated with 5-fluorouracil, CPT-11, and oxaliplatin at their respective IC₅₀s at several times. Cisplatin was also included in the analysis to investigate molecular mechanisms of varied efficacy and toxicity compared to oxaliplatin, with particular focus on oxaliplatin-induced peripheral neuropathy.

The inventors harvested cells at several time points ranging from 1-48 hours. The levels of p53 and two of its important downstream targets p21 and DR5 were measured in cell lysates via western blot. The variability in the kinetics of the p53 response in the wild-type cells was observed as early as one hour and continued out to forty-eight hours. Variability in the upregulation of p53 target genes was observed across treatment conditions that induced similar amounts of PARP cleavage and similar amounts of p53, suggesting that mechanistic differences separate from drug potency and level of p53 induction play a role in regulating classical p53 targets. Similar observations were made with two other p53 targets MDM2 and GADD45A, but not BAX.

These results show variability in the kinetics of the p53 response to different chemotherapeutic drugs. Based on the dramatic differences in regulation of these classical p53 targets at certain time points, the inventors assayed for whether this variability extended to much of the transcriptome.

### Example 4

Pan-Drug Gene Signature Contains Transcripts Critical to the Cellular Response to Chemotherapy in Colorectal Cancer Cells and Confirms Importance of p53 in Mediating this Response

After identifying significant differences in upregulation of p53 and four of its target genes across drugs at the protein level, the inventors evaluated this variability on a whole-transcriptome scale using microarrays.

To define a pan-drug signature and to evaluate heterogeneity in the p53 response on a whole-transcriptome scale, the inventors treated HCT116 and HCT116 p53^{-/-} cells with each of the four drugs at their IC₅₀ for eight hours. The RNA expression relative to an untreated control was measured using microarray analysis. Upregulation of p53 in these samples and equal induction of cell death at this dose and time point was validated by Western blot and CellTiterGlo, respectively, before microarray analysis. Internal control genes β-actin and GAPDH were evaluated, each of which showed no change across drug treatments. Each internal control had tightly clustered triplicates. Quality control measurements were also determined to be satisfactory for further analysis.

The inventors divided the master gene list into pan-drug, drug-specific, drug class-specific, p53-independent, and p53-dependent gene signatures. See an EXAMPLE below. Each complete signature, along with additional information for each gene was entered into the in the master list with the Probeset IDs, statistics, descriptions, etc.

The inventors used a filtering method to create p53-independent and p53-dependent gene signatures. See an EXAMPLE below.

### Example 5

Pan-Drug Gene Signatures after 5-Fluorouracil, CPT-11, Oxaliplatin, and Cisplatin Treatment of Human Colorectal Cancer Cells

A total of 961 transcripts were significantly up-regulated or down-regulated (fold change≥2 or ≤−2, p-value<0.05) by at least one drug and principal component analysis (PCA) mapping demonstrated clear separation between wild-type and p53^{-/-} cells. These 961 genes (the “master list”) were divided into pan-drug, drug-specific, or drug class-specific signatures which were further divided into p53-independent and p53-dependent signatures. The transcriptomic response to each drug varied greatly.

A total 36 transcripts were up-regulated or down-regulated (≥2 or ≤−2-fold change compared to the control, respectively) by all drug treatments. The pan-drug signature was further divided into p53-independent and p53-dependent signatures which revealed that most of the pan-drug transcriptomic response was p53-dependent. These results further support the understanding that p53 is a master regulator of the cellular response to chemotherapy. Genes within this pan-drug signature were regulated across drugs with various primary targets and mechanisms of action. These genes are likely fundamental to the response to chemotherapy. Thus, this signature may provide a set of prognostic biomarkers predicting response to chemotherapy.

By identifying fundamental elements of the p53 program in colorectal cancer, this signature may also direct the development of p53-reactivating compounds specifically more logically for treatment of colorectal cancer. Ten of the transcripts within the pan-drug signature were classified as “identified by AceView”, meaning that they have unknown function and sometimes unknown coding potential. One of these transcripts, skargluby, appeared at the top of the list, raising the possibility of regulated alternate expression.

B-cell translocation gene 2 (BTG2) was the most highly induced gene in the 5-fluorouracil, cisplatin, and oxaliplatin p53-dependent gene signatures and the second most highly induced gene in the CPT-11 p53-dependent signature.

GDF15, or growth differentiation factor 15, was also highly induced across all drug treatments. This TGF-beta ligand likely promotes epithelial to mesenchymal transition and metastasis in colorectal cancer through activation of Smad2 and Smad3 pathways. Evaluation of GDF15 in patients in several studies revealed that high levels correlate with increased chances of metastasis, lower overall survival, and weight loss. GDF15 neutralization in non-human primates decreased levels of cisplatin-induced weight. Conversely, 5-fluorouracil-resistant colorectal cancer cells express lower levels of GDF15 compared to 5-fluorouracil-sensitive cells and transient expression of GDF15 restores sensitivity, suggesting this gene plays an important role in 5-fluorouracil-mediated cell death. See Wang et al., Biomed. Res. Int., 2020, U.S. Pat. No. 2,826,010 (2020). GDF15 likely has a multiplex function in the response to chemotherapy and careful investigation is needed before combination therapies are considered.

Most of other transcripts in the pan-drug signature have a known tumor suppressive function. Others, such as PRDM1 and SESN1/2 may have complicated or multiplex functions in colorectal cancer and other cancers.

The results of this EXAMPLE indicate that the p53 response to chemotherapy includes genes that likely contribute to efficacy and toxicity and may contain transcripts that counteract anti-tumor effects of the drugs.

### Example 6

### Drug-Specific Signatures Suggest Novel Mechanisms of Efficacy and Toxicity Specific to 5-Fluorouracil, CPT-11, Oxaliplatin, or Cisplatin

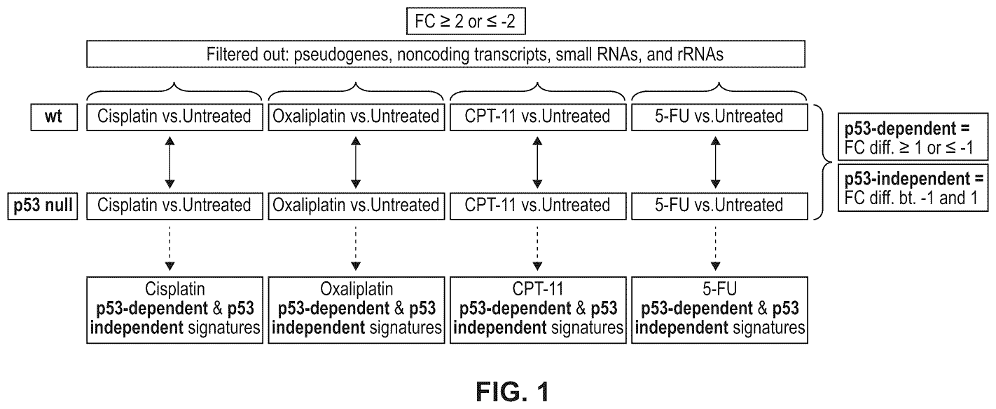

Most genes were regulated by 5-fluorouracil, CPT-11, oxaliplatin, and cisplatin in a drug-specific manner. The inventors further divided the drug-specific signatures were further divided into p53-independent and p53-dependent signatures using the filtering method in FIG. 1. Both p53-independent and p53-dependent signatures showed drug-specific effects that may indicate drug-specific mechanisms of efficacy.

The inventors then determined the transcripts differentially regulated by cisplatin and oxaliplatin to create cisplatin-specific and 5-fluorouracil/CPT-11/oxaliplatin-specific gene signatures. The 5-fluorouracil/CPT-11/oxaliplatin-specific signature contains transcripts that are upregulated by 5-fluorouracil, CPT-11, and oxaliplatin but are unaffected by cisplatin. Eight transcripts were uniquely regulated by platinum-based compounds.

The inventors found a strikingly unique down-regulation of histone genes by 5-fluorouracil both in a p53-independent and -dependent manner. This finding is supported by the results previous publications demonstrating that ionizing radiation-mediated DNA damage induces down-regulation of histone genes through the G1 checkpoint pathway, and that histone H2 levels can be regulated by 5-fluorouracil. The upregulation of histone proteins is needed in every round of cell replication. The downregulation of these genes by 5-fluorouracil may be a mechanism of efficacy of this drug.

The p53-dependent, CPT-11-specific regulation of transcripts included TLR3, a toll-like receptor known to promote anticancer immunity through activation of type I IFN; FDXR, or ferredoxin reductase, whose interaction with p53 is critical for tumor suppression via iron homeostasis; and DRAM1, a p53 target gene that modulates autophagy and apoptosis. Oxaliplatin uniquely upregulated SUSD2, which is commonly downregulated in colorectal cancer and interacts with the potential novel cytokine CSBF/C10orf99 to inhibit colorectal cancer cell growth, and SAT1, whose levels are also lower in patients with cancer and have a critical function in ferroptosis. Pan et al., Sci. Rep., 4, 6812 (2014); Ou, Wang, Li, Chu, Gu, Activation of SAT1 engages polyamine metabolism with p53-mediated ferroptotic responses. Proc. Natl. Acad. Sci. U.S.A., 113, E6806-12 (2016).

p53-independent and p53-dependent signatures also revealed previously unrecognized drug-specific effects that may indicate drug-specific mechanisms of toxicity. FOS, a commonly used marker of neuronal damage, was uniquely upregulated by oxaliplatin. FOS thus could function in oxaliplatin-induced peripheral neuropathy. Another group reported that oxaliplatin-treated mice exhibited neuronal damage (demonstrated by an upregulation of FOS and ATF3) that was reversible by treatment with metformin, which they suggest as a possible combination therapy to prevent/treat this side effect. Martinez et al., Neurobiol. Pain, 8, 100048 (2020); Pereira et al., Neurosci. Lett., 709, 134378 (2019). ATF3 appeared in a list of transcripts regulated differentially by cisplatin vs. oxaliplatin.

The dataset presented here help our understanding of why cisplatin has failed in the treatment of colorectal cancer patients, while similar platinum-based compounds like oxaliplatin have efficacy. While both these drugs rely on creation of adducts to halt DNA synthesis and repair, fewer adducts are needed for oxaliplatin to have a more potent effect. The cisplatin-specific mechanisms include upregulation of PRPF39, a pre-mRNA processing factor known to play a key role in sensitivity to cisplatin, downregulation of PITPNC1, which promotes metastasis-associated vesicular secretion, and downregulation of ROR1, whose high expression correlates with worse overall survival in colorectal cancer patients. Other transcripts in the cisplatin-specific signature with tumor-promoting function, including TBX3, which promotes epithelial to mesenchymal transition and predicts poor prognosis in colorectal cancer, and GBX2, which promotes growth of breast and prostate cancer cells.

Some transcripts in the signature containing genes that are upregulated by 5-fluorouracil, CPT-11, and oxaliplatin but that were unaffected by cisplatin can be used as biomarkers for worse outcomes (e.g., SERPINE1). These transcripts may have a dichotomous role in colorectal cancer (e.g., CEACAM1). Some transcripts in this signature (e.g., C1orf116, a driver of epithelial phenotype in epithelial-to-mesenchymal transition) may contribute to their unique efficacy.

The interplay of the transcripts within cisplatin-specific and 5-fluorouracil/CPT-11/oxaliplatin-specific signatures may explain the lack of cisplatin efficacy in treatment of colorectal cancer. The data presented here identify several candidate transcripts that may be especially important for an effective response to chemotherapy in colorectal cancer.

### Example 7

Subsets of Transcriptomic Signatures in Response to 5-Fluorouracil, CPT-11, Oxaliplatin, and Cisplatin Correlate with Patient Outcomes

The clinical relevance of these signatures was evaluated with The Cancer Genome Atlas (TCGA), which contains RNA-sequencing and microarray data on patient samples before treatment. Overall survival of colorectal adenocarcinoma patients was correlated with high vs. low (>1 or <−1 standard deviation from the mean, respectively) basal expression of transcripts within pan-drug, drug-specific, drug class-specific, p53-independent, and p53-dependent signatures. High vs. low basal expression of transcripts in the p53-dependent signatures were evaluated separately in p53 wild-type and p53 mutated patient groups, though this separation did not have a significant effect on correlation with overall survival. Existing literature was evaluated to supplement TCGA data in the establishment of these signatures. Nearly all signatures contained transcripts that correlated with overall survival.

TCGA and literature searching was used to establish subsets of gene signatures that correlate with patient outcomes. A logrank test p-value under 0.05 was considered significant, while a p-value between 0.05-0.2 was considered of potential interest. Both were included in the prognostic signatures. Genes within each signature that correlate with patient outcomes were listed along with representative Kaplan-Meier curves. The inventors organized the identity of these transcripts in the representative Kaplan-Meier curves.

The low basal expression of seventeen histone genes uniquely downregulated 5-fluorouracil correlated with improved overall survival. Only two histone genes that were downregulated by 5-FU correlated with improved survival when highly expressed. Many transcripts whose high expression correlated with improved survival were downregulated by the drugs, and vice versa. These are likely the mechanisms by which the drugs contribute to toxicity or resistance, suggesting that potential combination treatments to limit these effects should be investigated. These signatures can serve as prognostic biomarkers for colorectal cancer patients that are personalized based on p53 status.

### Example 8

Cytokines TRAILR2, IL-8, VEGF, and Ferritin are Regulated Differently Across Treatments with 5-Fluorouracil, CPT-11, Oxaliplatin, Cisplatin, and Clinically Relevant Combinations

Chemotherapy increases amounts of circulating cytokines. Optimizing this induction for both identity and magnitude is necessary for maximizing the anti-tumor immune effects of chemotherapy and avoiding cytokine storm. Some prior publications have evaluated the effects of 5-fluorouracil, CPT-11, oxaliplatin, and cisplatin on cytokine levels, but not directly across all four drugs, clinically relevant combinations, and p53 status.

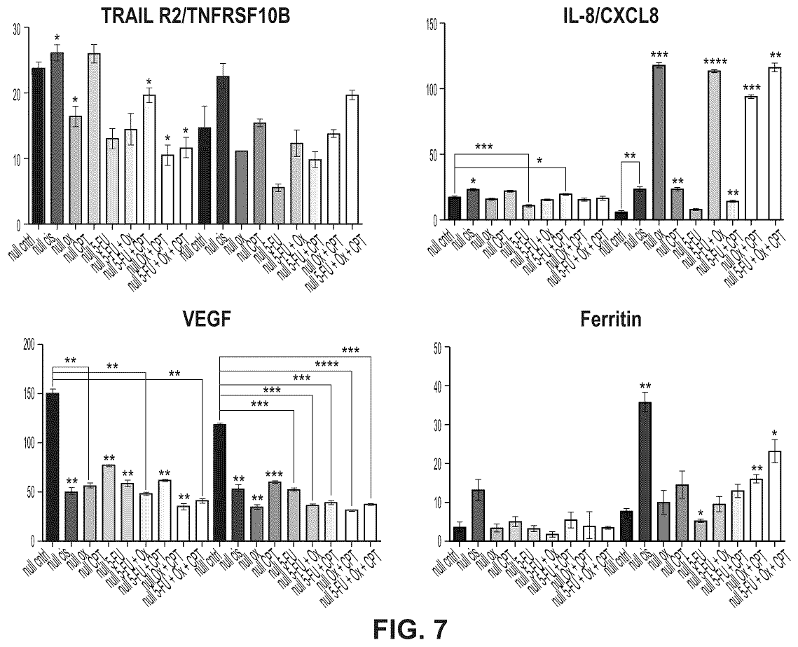

HCT116 and HCT116 p53^{-/-} cells were treated with the four drugs (5-FU, CPT-11, oxaliplatin, and cisplatin) at their IC50s and combination treatment groups received two-three drugs, each at their individual IC₅₀ concentration. Cytokine levels in the cell supernatants were measured with the Luminex 200 platform and significant differences between control and treated groups were noted for cytokines TRAILR2, IL-8, VEGF, and ferritin. See FIG. 7.

Soluble TRAILR2 (death receptor 5; DR5) is a decoy receptor for TRAIL, an apoptosis-inducing cytokine. Using the method identified that TRAILR2 was down-regulated by oxaliplatin and 5-fluorouracil, was not affected by CPT-11, and was increased by cisplatin. TRAILR2 levels were lower in the oxaliplatin and 5-fluorouracil treated cells compared to cisplatin and CPT-11 treated cells. The effect was not synergistic. Downregulation of TRAILR2 by oxaliplatin seemed dependent on p53, but p53-independent by 5-fluorouracil. Cisplatin upregulated TRAILR2 despite p53 status, suggesting a conflicting mechanism between cisplatin, oxaliplatin, and 5-FU not reported previously. Using the method identified an increase in IL-8 by oxaliplatin and an increase in ferritin by cisplatin which may contribute to cancer cell survival. 5-Fluorouracil, CPT-11, and oxaliplatin, and cisplatin, downregulated VEGF production by treatment of colorectal cancer cells. Soluble TRAILR2 may provide a readout on a mechanism by which certain tumors may evade the innate immune system.

Upregulation of IL-8, a pro-inflammatory cytokine thought to have an immunosuppressive effect in the tumor microenvironment and whose levels in serum correlate with colorectal cancer progression, was drastically different across p53 status. See Bakouny & Choueiri, Nature Medicine, 26, 650-51 (2020); David, Dominguez, Hamilton, & Palena, Vaccines (Basel), 4, 22 (2016). Oxaliplatin did not affect IL-8 levels in the wild-type cells, but induced IL-8 the p53^{-/-} cells. This finding could have a significant impact on how oxaliplatin is administered to patients based on their p53 status. CPT-11 single agent treatment and CPT-11+5-FU treatment also caused a moderate increase in IL-8 despite p53 status.

Vascular endothelial growth factor (VEGF) is a pro-angiogenic cytokine responsible for supplying oxygen and nutrients to the tumor and promoting cancer cell escape. All four drugs significantly downregulated release of VEGF by both HCT116 and HCT116^{-/-} cells with no apparent synergistic effect of drug combinations.

Ferritin is recognized in the medical art to have several tumor-promoting effects including protection of cancer cells from reactive oxygen species and promoting the pro-tumorigenic M2 program in macrophages. Alkhateeb & Connor, Biochim. Biophys. Acta, 1836, 245-54 (2013). Ferritin is used as a prognostic marker in some cancers. Lee, Jeon, & Shim, J. Cancer, 10, 1717-25 (2019). Increased ferritin expression also limits ferroptosis, an iron-dependent form of cell death distinct from apoptosis. In wild-type cells, no drug significantly affected ferritin levels. In p53^{-/-} cells, there was a large increase in ferritin after cisplatin treatment. Interestingly, oxaliplatin plus CPT-11 and 5-fluorouracil plus oxaliplatin and CPT-11 treatment increased ferritin levels more so than single treatments, though this effect does not seem to be synergistic. No notable changes were observed in GM-CSF, C-reactive protein, CXCL13, IL-18, CCL22, or IFN-alpha profiles.

Together, these data suggest that chemotherapeutics and p53 status can have a large impact on cytokine regulation. Most notable findings include an increase in IL-8 by oxaliplatin and increase in ferritin by cisplatin. As both cytokines are thought to contribute to cancer cell survival, care should be taken in administering specific therapies to patients based on their basal expression of these cytokines and the p53 status of the tumors.

### Example 9

### Non-Prognostic Gene Signatures in Colorectal Cancer

Non-prognostic colorectal cancer signatures have prognostic value in breast cancer. Using drug-induced gene signatures established in EXAMPLES above, the inventors established non-prognostic (P-value>0.2) CRC gene signatures using cBioPortal. While these signatures were non-prognostic in colorectal cancer, several transcripts were prognostic (P-value<0.05) in breast (underlined, bolded text).

TABLE 1
Non-prognostic gene signatures in colorectal cancer

| | | |
|---|---|---|
| Pan-drug | FDXR | EGR1 |
| ATF3 | FHIT | ETS1 |
| BTG2 | FMNL2 | FAM214A |
| CD274 | FTO | FGD1 |
| DCP1B | G2E3 | FGD4 |
| E2F7 | GRHL3 | FOS |
| IER5 | HMGA2 | GLIPR1 |
| MDM2 | HS6ST2 | HOXB8 |
| PLK2 | IFIT3 | HS6ST1 |
| POLH | IFNAR2 | KLF6 |
| PRDM1 | IKBIP | KLK6 |
| RNF19B | IPO11; LRRC70 | KLK7 |
| SERTAD1 | IRS2 | KRTAP2-2 |
| SESN1 | ISG15 | LAMB3 |
| TOB1 | KIAA0040 | MAP1S |
| TP53INP1 | KITLG | MBD5 |
| TUBGCP3 | KSR2 | NKTR |
| 5-FU-specific | LAMP3 | NR4A3 |
| ARHGEF39 | LIN37 | NUAK1 |
| CCNG2 | LRBA | RBM24 |
| CENPA | LYNX1 | RIOK3 |
| DTL | MED13L | RNU11 |
| FAM83D | MGAT5 | SAT1 |
| HIST1H2AH (H2AC12) | MSI2 | SHFM1 (SEM1) |
| HIST1H2AJ (H2AC14) | NBAS | SRSF5 |
| HIST1H2BB (H2BC3) | NTPCR | SUSD2 |
| HIST1H2BF (H2BC7) | OR51B5 | TGM2 |
| HIST1H2BH (H2BC9) | OR51I1 | THEM6 |
| HIST1H2BK (H2BC12) | PARD6G | TIGAR |
| HIST1H2BO (H2BC17) | PDLIM3 | TUFT1 |
| HIST1H3A (H3C1) | PIBF1 | ULBP1 |
| HIST1H3B (H3C2) | PPP3CA | VCAN |
| HIST1H3F (H3C7) | PRRG4 | Cisplatin-specific |
| HIST1H3G (H3C8) | PTCH1 | ADAM22 |
| HIST1H4A (H4C1) | PTK2 | ARHGEF3 |
| HIST1H4B (H4C2) | PTPRO | ARID5B |
| HIST1H4D (H4C4) | RAB27B | ATXN1 |
| HIST1H4K (H4C12) | RAB33B | BAZ2B |
| HIST2H2AB (H2AC21) | RABGAP1L | BTBD9 |
| HIST2H2AC (H2AC20) | RAD51B | CDKL5 |
| HIST3H2A (H2AW) | RAET1G | CMIP |
| INCENP | RASA1 | DOCK4 |
| KIRREL | REEP1 | EXT1 |
| MYBL1 | RGS5 | FAM217B |
| PLAU | RHOBTB3 | FUT8 |
| PSMC3IP | RINL | GBX2 |
| RASSF6 | RPS27L | GTF2IRD1 |
| SOX4 | RRM2B | IGF2BP3 |
| TRAF4 | RSRC1 | JARID2 |
| TRIM59 | SDC1 | KAT6B |
| TTK | SFN | KDM4C |
| ZNF658 | SLC29A3 | KLF12 |
| CPT-11-specific | SMAD3 | KLHL29 |
| ABCA12 | SPAG5 | MAML2 |
| AKR1B15 | STAT4 | MYO1E |
| APAF1 | STK17A | NFKBIA |
| APOBEC3C | STK39 | NR6A1 |
| APOBEC3H | TBL1X | OPHN1 |
| ARHGAP10 | TCP11L1 | OR2M3 |
| BAG1 | THUMPD3-AS1 | OR8D1 |
| BCKDHB | TLR3 | PHLPP1 |
| BIRC3 | TMEM63B | PITPNC1 |
| CAMK2D | TNFRSF10C | PRKCE |
| CCDC91 | TP53I3 | PRPF39 |
| CCNB2 | TP73 | PTPRM |
| CDKAL1 | TRIO | SIPA1L3 |
| CDKL2 | TRPM6 | SSH2 |
| CITED2 | VPS13B | TANC1 |
| CMBL | VTI1A | TANC2 |
| CNNM4 | WDR63 (DNAI3) | TBC1D22A |
| COG5 | XPC | TBX3 |
| COL17A1 | XRCC4 | TESK1 |
| CTBP2 | ZNF672 | TIAM1 |
| DEPDC7 | ZNF804A | TMEM133 |
| DIAPH2 | ZNF823 | (ARHGAP42) |
| DOCK3 | Oxaliplatin-specific | TNS3 |
| DRAM1 | AEN | TSPAN18 |
| DST | AHNAK2 | UVRAG |
| DYM | C3orf14 | ZNF407 |
| EFNB1 | CD22; MIR5196 | ZNF426 |
| EHBP1 | CSRNP1 | CisOx-specific |
| EPN3 | CTSS | ARRDC4 |
| EXOC6B | DUSP1 | GADD45A |
| FAF1 | DUSP19 | LPAR6 |
| | | SLC10A5 |
| | | ZRSR2 |

### Other Embodiments

Specific compositions and methods for optimizing the treatment of colorectal cancer have been described. The scope of the invention should be defined solely by the claims. A person having ordinary skill in the biomedical art interprets all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

### CITATION LIST

A person having ordinary skill in the biomedical art can use these patents, patent applications, and scientific references as guidance to predictable results when making and using the invention.
Alcindor & Beauger, Oxaliplatin: a review in the era of molecularly targeted therapy. Curr. Oncol., 18, 18-25 (2011).
Alkhateeb & Connor, The significance of ferritin in cancer: Anti-oxidation, inflammation, and tumorigenesis. Biochim. Biophys. Acta, 1836, 245-54 (2013).
Bailly, Irinotecan: 25 years of cancer treatment. Pharmacol. Res., 148, 104398 (2019).
Bakouny & Choueiri, IL-8 and cancer prognosis on immunotherapy. Nature Medicine, 26, 650-51 (2020).
Botcheva & McCorkle, Cell context dependent p53 genome-wide binding patterns and enrichment at repeats. PLoS One, 9(11), el 13492 (Nov. 21, 2014).
Bunz et al., Disruption of p53 in human cancer cells alters the responses to therapeutic agents. J. Clin. Invest., 104(3), 263-9 (August 1999).
Burns & El-Deiry, Microarray analysis of p53 target gene expression patterns in the spleen and thymus in response to ionizing radiation. Cancer Biol Ther. 2003 July-August; 2(4):431-43.
Burns, Bernhard, & El-Deiry, Tissue specific expression of p53 target genes suggests a key role for KILLER/DR5 in p53-dependent apoptosis in vivo. Oncogene 20, 4601-4612 (2001).
Carlsen & El-Deiry, W. S. Differential p53-Mediated cellular responses to DNA-damaging therapeutic agents. International Journal of Molecular Sciences, 22(21), 11828 (2021). The contents of this publication are specifically incorporated by reference.
Carlsen et al., Pan-drug and drug-specific mechanisms of 5-FU, irinotecan (CPT-11), oxaliplatin, and cisplatin identified by comparison of transcriptomic and cytokine responses of colorectal cancer cells. Oncotarget, 12(20), 2006-2021 (2021). The contents of this publication are specifically incorporated by reference.
Carlsen, Huntington, & El-Deiry, Immunotherapy for colorectal cancer: Mechanisms and predictive biomarkers. Cancers, 14(4), 1028 (2022). The contents of this publication are specifically incorporated by reference.
Chen et al. P53 status as a predictive biomarker for patients receiving neoadjuvant radiation-based treatment: a meta-analysis in rectal cancer. PLoS One, 7(9):e45388 (2012).
Current Protocols in Immunology (CPI) (2003). John E. Coligan, ADAM Kruisbeek, David H Margulies, Ethan M Shevach, Warren Strobe, (eds.) John Wiley and Sons, Inc.
Current Protocols in Molecular Biology (CPMB), Frederick M. Ausubel ed. (John Wiley and Sons, 2014).
Current Protocols in Protein Science (CPPS), John E. Coligan, ed. (John Wiley and Sons, Inc., 2005).
David, Dominguez, Hamilton, & Palena, The IL-8/IL-8R axis: A double agent in tumor immune resistance. Vaccines (Basel), 4, 22 (2016).
Davis et al., Basic Methods in Molecular Biology (Elsevier Science Publishing, Inc., New York, USA, 2012).
Dean, Irinotecan therapy and UGT1A1 genotype [Updated Apr. 4, 2018]. In: Pratt et al., editors. Medical Genetics Summaries [Internet]. (Bethesda, MD, USA: National Center for Biotechnology Information (US); 2012).
Del Rio et al., Gene expression signature in advanced colorectal cancer patients select drugs and response for the use of leucovorin, fluorouracil, and irinotecan. J. Clin. Oncol., 25, 773-80 (2007).
Dempke & Heinemann, Ras mutational status is a biomarker for resistance to EGFR inhibitors in colorectal carcinoma. Anticancer Res., 30(11), 4673-7 (November 2010).
Encyclopedia of Molecular Cell Biology and Molecular Medicine, Robert S. Porter et al., eds. (Blackwell Science Ltd., 1999-2012).
Encyclopedia of Molecular Cell Biology and Molecular Medicine, Robert S. Porter et al., eds. (Blackwell Science Ltd., 1999-2012).
Fischer, Census and evaluation of p53 target genes. Oncogene, 36, 3943-56 (2017).
Green & Sambrook, Molecular Cloning: A Laboratory Manual, 4th ed., (Cold Spring Harbor Laboratory Press, Cold Spring Harbor, N.Y., USA 2012).
Groysman et al., Chemotherapy-induced cytokines and prognostic gene signatures vary across breast and colorectal cancer. American Journal of Cancer Research, 11(12), 6086-6106 (2021). The contents of this publication are specifically incorporated by reference.
Immunology, Werner Luttmann, ed. (Elsevier, 2006).
Janeway's Immunobiology, Kenneth Murphy, Allan Mowat, Casey Weaver, eds., (Taylor & Francis Limited. 2014).
Laboratory Methods in Enzymology: DNA, Jon Lorsch, ed. (Elsevier, 2013).
Lee, Jeon, & Shim, Prognostic value of ferritin-to-hemoglobin ratio in patients with advanced non-small-cell lung cancer. J. Cancer, 10, 1717-25 (2019).
Lewin's Genes XI (Jones & Bartlett Publishers, 2014).
Martinez et al., Metformin protects from oxaliplatin induced peripheral neuropathy in rats. Neurobiol. Pain, 8, 100048 (2020).
Merck Manual of Diagnosis and Therapy, 19^{th} edition, (Merck Sharp & Dohme Corp., 2011)
Molecular Biology and Biotechnology: A Comprehensive Desk Reference, Robert A. Meyers, ed. (VCH Publishers, Inc., 1995).
Molecular Biology and Biotechnology: A Comprehensive Desk Reference, Robert A. Meyers, ed. (VCH Publishers, Inc., 1995).
Mukherji et al., Three different polymorphisms of the DPYD gene associated with severe toxicity following administration of 5-FU: a case report. J. Med. Case Reports 13, 76 (2019).
Negrei et al., Colon cancer cells gene expression signature as response to 5-fluorouracil, oxaliplatin, and folinic acid treatment. Front. Pharmacol., 7, 172 (2016).
Nikulenkov et al., Insights into p53 transcriptional function via genome-wide chromatin occupancy and gene expression analysis. Cell Death Differ., 19(12), 1992-2002 (December 2012).
Ou, Wang, Li, Chu, Gu, Activation of SAT1 engages polyamine metabolism with p53-mediated ferroptotic responses. Proc. Natl. Acad. Sci. U.S.A., 113, E6806-12 (2016).
Pan et al., CSBF/C10orf99, a novel potential cytokine, inhibits colon cancer cell growth through inducing G1 arrest. Sci Rep. 2014; 4:6812.
Pereira et al., Metformin reduces c-Fos and ATF3 expression in the dorsal root ganglia and protects against oxaliplatin-induced peripheral sensory neuropathy in mice. Neurosci. Lett., 709, 134378 (2019).
Pharmaceutical Sciences 23^{rd} edition (Elsevier, 2020).
Smeenk et al., Role of p53 serine 46 in p53 target gene regulation. PLoS One, 6(3), e17574 (Mar. 4, 2011).
Wang et al., GDF15 repression contributes to 5-fluorouracil resistance in human colon cancer by regulating epithelial-mesenchymal transition and apoptosis. Biomed. Res. Int., 2020, U.S. Pat. No. 2,826,010 (2020).
Wyatt & Wilson, 3rd, Participation of DNA repair in the response to 5-fluorouracil. Cell. Mol. Life Sci., 66, 788-99 (2009).
Yuniati, Scheijen, van der Meer, & van Leeuwen. Tumor suppressors BTG1 and BTG2: Beyond growth control. J. Cell. Physiol., 234, 5379-89 (2019).
Zhang et al., Advanced strategies for therapeutic targeting of wild-type and mutant p53 in cancer. Preprints, 2022, 2022010020 (2022)
Zhang, Yin, Xu, & Chen, 5-Fluorouracil: Mechanisms of resistance and reversal strategies. Molecules, 13, 1551-69 (2008).

All patents and publications cited throughout this specification are expressly incorporated by reference to disclose and describe the materials and methods that might be used with the technologies described in this specification. The publications discussed are provided solely for their disclosure before the filing date. They should not be construed as an admission that the inventors may not antedate such disclosure under prior invention or for any other reason. If there is an apparent discrepancy between a previous patent or publication and the description provided in this specification, the present specification (including any definitions) and claims shall control. All statements as to the date or representation as to the contents of these documents are based on the information available to the applicants and constitute no admission as to the correctness of the dates or contents of these documents. The dates of publication provided in this specification may differ from the actual publication dates. If there is an apparent discrepancy between a publication date provided in this specification and the actual publication date supplied by the publisher, the actual publication date shall control.

The foregoing written specification is considered sufficient to enable one skilled in the biomedical art to practice the present aspects and embodiments. The present aspects and embodiments are not to be limited in scope by examples provided, since the examples are intended as a single illustration of one aspect and other functionally equivalent embodiments are within the scope of the disclosure. Various modifications besides those shown and described herein will become apparent to those skilled in the biomedical art from the foregoing description and fall within the scope of the appended claims. The advantages and objects described herein are not necessarily encompassed by each embodiment. Those skilled in the biomedical art will recognize or be able to ascertain using no more than routine experimentation, many equivalents to the specific embodiments described herein. Such equivalents are intended to be encompassed by these claims.

1. A method for optimizing the treatment of gastrointestinal cancer in a subject comprising the steps of
(a) obtaining gastrointestinal cancer cells extracted from the subject,
(b) determining the transcriptional response in gastrointestinal cancer cells across four or more clinically used chemotherapeutic drugs in terms of gene identity, magnitude of change, and p53 dependence, and
(c) determining the gene signature to determine the optimal course of treatment for the subject.

2. The method of claim 1, further comprising the step of:
(d) administering to the patient a therapeutically effective amount of 5-fluorouracil, irinotecan, oxaliplatin, or a combination thereof.

3. The method of claim 1, wherein the gastrointestinal cancer is colorectal cancer.

4. The method of claim 1, wherein the determination of the transcriptional response produces both p53-independent, and p53-dependent signatures.

5. The method of claim 1, wherein the determination of the transcriptional response produces a measurement of the regulation of histone genes by chemotherapeutic drug, such that a downregulation of histone genes correlates with improved survival in gastrointestinal cancer patients.

6. The method of claim 1, wherein the determination of the transcriptional response produces a measurement of the regulation of FOS and ATF3 genes by chemotherapeutic drug, such that a upregulation of FOS and ATF3 correlates with peripheral neuropathy.

7. The method of claim 1, further comprising the step of:
(d) administering to the patient a therapeutically effective amount of an anti-BTG2 therapeutic.
